(19) United States
(12) Patent Application Publication
(10) Pub. No.: US 2026/0137652 A1
(43) Pub. Date: May 21, 2026

(54) Composition Comprising Non-Phosphorylated Tryptamine

(71) Applicant: SINTALICA S.R.L., Arezzo (IT)

(72) Inventors: Luca Santi, Rome (IT); Luigi Mondello, Messina (IT); Anna Notti, Messina (IT); Illaria Cacciotti, Rome (IT); Salvatore Cuzzocrea, Messina (IT); Alessia Tropea, Messina (IT)

(21) Appl. No.: 19/119,717

(22) PCT Filed: Oct. 11, 2023
(86) PCT No.: PCT/IB2023/060208

§ 371 Date: Apr. 9, 2025

(30) Foreign Application Priority Data
Oct. 11, 2022 (IT) 202200020892

(51) Int. Cl. A61K 31/4045; A61K 31/01; A61K 31/015; A61K 31/122; A61K 31/198; A61K 31/375; A61K 31/405; A61K 31/4172; A61K 31/437; A61K 31/4415; A61K 31/522; A61K 36/07; A61P 29/00

(52) U.S. Cl. CPC A61K 31/4045; A61K 31/01; A61K 31/015; A61K 31/122; A61K 31/198; A61K 31/375; A61K 31/405; A61K 31/4172; A61K 31/437; A61K 31/4415; A61K 31/522; A61K 36/078; A61P 29/00

(57) ABSTRACT

The present invention relates to a composition comprising at least one psychedelic non-phosphorylated tryptamine, preferably derived from psychedelic psilocybin mushrooms, more preferably derived from psychedelic psilocybin mushrooms belonging to the Psilocybe genus, at least one antioxidant, wherein the antioxidant is at least one carotenoid, and at least one supplement selected from an amino acid, a vitamin, theobromine, caffeine, resveratrol, gamma aminobutyric acid and combinations thereof. The invention relates also to the pharmaceutical uses of the composition of the invention.

### REFERENCE TO RELATED APPLICATIONS

This is a national phase application of PCT/IB2023/0060208, the disclosure of which is hereby incorporated by reference.

### FIELD OF THE INVENTION

The present invention relates to a composition comprising at least one psychedelic non-phosphorylated tryptamine, at least one antioxidant, wherein the antioxidant is at least one carotenoid, and at least one supplement selected from an amino acid, a vitamin, theobromine, caffeine, resveratrol, gamma aminobutyric acid and combinations thereof. The invention relates also to the pharmaceutical uses of the composition of the invention.

### BACKGROUND OF THE INVENTION

In recent years, the scientific interest towards the potential use of psilocybin and other psychedelics for medical applications, such as the psychiatric disorders treatment, including mood disorders, depression, anxiety and alcoholism and nicotine addiction is increasing (Rucker, J. J. H et al., “Psychiatry & psychedelic drugs. Past, present & future”, Neuropharmacology (2018), v. 142, p. 200-218).

Psilocybin (4-phosphoryloxy-N,N-dimethyltryptamine) is a substituted indolealkylamine and belongs to the group of hallucinogenic tryptamines (tryptamine alkaloid). Psilocybin is isolated from various genera of fungi including the genus Psilocybe, which is known to have hallucinogenic, anxiolytic, and psychoactive activities.

As well known, psilocybin is a prodrug: in fact, when it is orally administered, while passing through the liver it undergoes dephosphorylation to psilocin active drug, which can cross the blood-brain barrier and produce its psychoactive effects. Psilocin binds serotonin 2A (5-HT2A) receptors in the central nervous system (CNS), mimicking the effects of serotonin. The serotonin 2A receptors (5-HT2A) are implicated in mental disorders with complex etiologies and in physiological processes such as learning and memory and in neurogenesis. The activation of these receptors by psychedelic compounds, such as psilocybin and psilocin has been shown to provide benefit in therapies that address mental health disorders. However, due to rapid metabolism and clearance of psilocin in the body by both monoamine oxidase (MAO) and aldehyde dehydrogenase (ALDH) to 4-hydroxy-indole-3-acetaldehyde, further metabolized to 4-hydroxyindole-3-acetic acid, only a small amount can act on a serotonin receptor. As a result, there is a need to provide improved methods and compositions to inhibit the metabolic breakdown and clearance of a psychedelic compound, such as psilocin, by the monoamine oxidase (MAO) as well as to improve the tryptamine compositions to synergistically modulate the activity of the physiological response to the psychedelic compounds because of the activation of a serotonin receptor.

Monoamine oxidases (MAOs) are metabolic enzymes attached to cytosolic side of the outer membrane of mitochondria of neuronal, glial and several cell types. Specifically, they catalyze the oxidative deamination of neuroactive and vasoactive biogenic compounds (including serotonin and tryptamines) and xenobiotic amines into the corresponding aldehyde and ammonia, both in the central nervous system and peripheral tissues. Several monoamine oxidase inhibitors (MAOIs) have been extensive employed as antidepressants and neuroprotective agents in Parkinson's disease, as well as in the treatment of anxiety. There are two main isoforms, MAO-A and MAO-B. Monoamine oxidase A is predominantly responsible for the metabolism of psilocin (Reniers et al., “Synthesis and evaluation of 3-carboline derivatives as potential monoamine oxidase inhibitors”, Bioorg. Med. Chem. (2011) v. 19(1), p. 134-44). Non-phosphorylated tryptamines such as psilocin and norpsilocin, which can be extracted from psychedelic psilocybin mushrooms, for example from Psilocybe mushrooms, are highly unstable: in fact, they can degrade due to chemical and physical ambient conditions, e.g., heat, light, water, and/or oxygen.

For example, psilocin is highly unstable and degrades quickly in the presence of air, heat, and/or light and in solution. Degradation of natural psychedelic products especially occurs during extraction procedures. During the extraction process, in fact, they can degrade over time in different aqueous and ethanol solutions, as fluid solutions, dispersions, tinctures, emulsion, and powdered extractions. Natural psychedelic product can degrade over time in current delivery dose forms after manufacturing, during storage, and before use.

Oxidation is one of the chemical degradation pathways that can occurs; thus, to avoid this reaction the stabilizer may be an antioxidant, capable to protect the active ingredient from “free radicals”, as disclosed in US2021353615.

It is predicted that alternative brain receptor transmitters, such as those replacing or reducing serotonin receptor uptake, and or hallucinogens, such as Ayahuasca, dimethyltryptamine (DMT), and or psilocin/psilocybin extracts or mushroom exposure may be positively enhanced in the presence of selected supplements and may more effectively potentiate a positive experience, and further potentiate multiple medical benefits, including but not limited to psychosocial, anti-depressive, panic or post-traumatic stress related, general anxiolytic, and with frequent or daily use facilitate cognitive enhancement, greater amelioration of one or more dementias (US20210267985).

Despite the above solutions, it remains the need for compositions which are stable and useful for the enhancement of cognitive, emotional and/or perceptual functions; or for promoting neurogenesis, resolving neuropathy, and/or improving neurological health.

### DEFINITIONS

Unless otherwise defined, all the terms of the art, notations and other scientific terms used herein are intended to have the meanings commonly understood by those who are experts in the technique to which this description belongs. In some cases, terms with commonly understood meanings are defined here for clarity and I or for ready reference; the inclusion of these definitions in this description should therefore not be interpreted as representing a substantial difference with respect to what is generally understood in the art.

The terms “comprise”, “have”, “include”, “contain”, “comprising”, “having”, “including” and “containing” are to be understood as open terms (i.e. the meaning “comprising, but not limited to”) and are to be considered as a support also for terms such as “essentially consist of”, “consisting essentially of”, “consist of” or “consisting of”.

For all the ranges indicated in the text and in the claims of the present patent application, it is understood that the extremes of these ranges are included.

The terms “psychedelic drugs” or “psychedelic compounds” or “psychedelics” are synonymous, and they mean classes including tryptamines (“psychedelic tryptamines”), phenethylamines, and lysergamides. Some of the psychedelic drugs being researched for therapy include psilocybin, psilocin LSD (lysergic acid diethylamide), DMT (dimethyltryptamine), ibogaine, mescaline, and MDMA (3,4-methylenedioxymethamphetamine).

The “psychedelic compounds”, in particular the psychedelic non-phosphorylated tryptamines used in the invention, are preferably derived from psychedelic psilocybin mushrooms, more preferably derived from psychedelic psilocybin mushrooms belonging to the Psilocybe genus.

The psychedelic psilocybin mushrooms include a polyphyletic, informal group of fungi that contain psilocybin, psilocin, or both within their biomass, typically within their fruiting bodies, resulting in their activation of a psychedelic reaction in a subject

Preferred psychedelic compounds used in the invention comprise non-phosphorylated tryptamines, such as psilocin and/or norpsilocin, their derivatives and/or combination thereof.

Also comprised in the definition of said psychoactive non-phosphorylated tryptamine are the extracts from psychedelic psilocybin mushrooms, preferably belonging to the Psilocybe genus, whereas said extract has been purified to contain only non-phosphorylated tryptamines.

The pharmaceutically acceptable salts, derivatives, hydrate, or solvate of the above cited psychedelic compounds are comprised in the definition too.

The term “Psilocybe genus” may refer to the following non-limiting examples of suitable mushrooms containing psilocybin-like psychedelic compounds: Psilocybe atlantis, Psilocybe azurenscens, Psilocybe bohemica, Psilocybe baeocystis, Psilocybe cyanescens, Psilocybe cubensis, Psilocybe tampanensis, Psilocybe hoogshagenii Psilocybe mexicana, Psilocybe ovoideocystidiata, Psilocybe semilanceata, Psilocybe weraroa, Psilocybe stuntzii, Psilocybe cyanofibrillosa, Psilocybe zapotacorum, Psilocybe yungensis, Psilocybe liniformans, Psilocybe xalapensis, Psilocybe venenata, Psilocybe subtropicalis, Psilocybe singer, Psilocybe schultesii, Psilocybe rostrata, Psilocybe quebecensis, Psilocybe pintonii, Psilocybe puberula, Psilocybe mairei, Psilocybe laurae, Psilocybe kumaenorum, Psilocybe beheimii, Psilocybe begalindoi, Psilocybe fmetaria, Psilocybe beegonii, Psilocybe dumontii, Psilocybe carbonaria, Psilocybe cordispora, Psilocybe bispora, Psilocybe aucklandii, and combinations thereof.

The term “pharmaceutically acceptable salts or derivatives” herein refers to those salts or derivatives which possess the biological effectiveness and properties of the salified or derivatized compound and which and which do not produce adverse reactions when administered to a mammal, preferably a human. The pharmaceutically acceptable salts may be inorganic or organic salts; examples of pharmaceutically acceptable salts include but are not limited to carbonate, hydrochloride, hydrobromide, sulphate, hydrogen sulphate, citrate, maleate, fumarate, trifluoroacetate, 2-naphthalenesulphonate, and para-toluenesulphonate. Further information on pharmaceutically acceptable salts can be found in Handbook of pharmaceutical salts, P. Stahl, C. Wermuth, WILEY-VCH, 127-133, (2008), herein incorporated by reference. The pharmaceutically acceptable derivatives include the esters, the ethers, and the N-oxides.

“Psilocybin” is the common name of 4-phosphoryloxy-N,N-dimethyltryptamine.

“Psilocin” is the common name of 4-hydroxy-N,N-dimethyltryptamine.

“Baeocystin” is the common name of 4-phosphoryloxy-N-methyltryptamine.

“Norpsilocin” is the common name of 4-hydroxy-N-methyltryptamine. “Norbaeocystin” is the common name of 4-Hydroxytryptamine 4-phosphate.

“Aeruginascin” is the common name of N,N,N-trimethyl-4-phosphoryloxytryptamine.

The “carotenoids” also called “tetraterpenoids”, are yellow, orange, and red organic pigments that are produced by plants and algae, as well as several bacteria, and fungi. Carotenoids can be further categorized into two classes, xanthophylls (which contain oxygen) and carotenes (which are purely hydrocarbons and contain no oxygen). Examples of carotenes are a-carotene, β-carotene, lycopene, astaxantin and andzeaxantine. All carotenoids are derivatives of tetraterpenes, meaning that they are produced from 8 isoprene molecules and contain 40 carbon atoms. In general, carotenoids absorb wavelengths ranging from 400 to 550 nanometers (violet to green light). Carotenoids that contain unsubstituted beta-ionone rings (including 3-carotene, a-carotene, p-cryptoxanthin, and y-carotene) have vitamin A activity (meaning that they can be converted to retinol).

Preferably, the carotenoids used in the present invention are obtained by plants extraction and/or microbial fermentation.

The “xanthine derivatives” are agents that resemble natural occurring xanthines such as caffeine, theobromine and methylxanthines (such as theophylline), which are included in the meaning of the present invention. The term “MAOIs” means monoamine oxidases inhibitors. Preferred MAOIs belong to the p-carboline class of inhibitors. Preferred p-carboline compounds are harman, harmine, norharmane, harmol, 6-methoxyharmalan, harmalan, harmaline, harmalol, dihydro-p-carbolines (DHpC), tetrahydro-3-carboline (THpC), tetrahydroharmine, methyl-tetrahydro-p-carboline MTHpC, pinoline, 1-trichloromethyl-1,2,3,4tetrahydro-b-carboline (TaClo), 6-methoxytetrahydroharmalan, ethyl P-carboline-3-carboxylate (PCCE), p-carboline-3-carboxylate (PCCM), manzamine A, manzamine X, 6-deoxymanzamine X, manzamine Y, 8-hydroxymanzamine A, 8-methoxymanzamine A, 6-hydroxymanzamine A, 3,4-dihydromanzamine A, ent-8-hydroxymanzamine A, ent-manzamine F, neo-kauluamine, xestomanzamine B, hyrtioerectines A, gesashidine A, plakortamines A, plakortamines B, plakortamines D, plakortamines C, eudistomidins, threctandramine, fascaplysin and/or salts, derivatives, hydrate, or solvate thereof and/or combination thereof.

The acronym “CAR” means carrageenan.

The acronym “MPO” means myeloperoxidase.

The abbreviation “CPX” means complex.

The abbreviation “VEH” means vehicle.

### SUMMARY OF THE INVENTION

The present invention relates to a composition comprising (a) at least one psychedelic non-phosphorylated tryptamine, (b) at least one antioxidant, wherein the antioxidant is at least one carotenoid, and (c) at least one supplement selected from an amino acid, a vitamin, theobromine, caffeine, resveratrol, gamma aminobutyric acid and combinations thereof.

A further object of the invention relates to the composition comprising (a) at least one psychedelic non-phosphorylated tryptamine, (b) at least one antioxidant comprising at least one carotenoid, and (c) at least one supplement selected from an amino acid, a vitamin, theobromine, caffeine, resveratrol, gamma aminobutyric acid and combinations thereof, for use in the treatment of fibromyalgia, spinal cord injury-induced chronic neuropathic pain, neuropathic pain associated with diabetic peripheral neuropathy, post-herpetic neuralgia, chronic musculoskeletal pain and/or a TNF-α-induced inflammatory disease. According to a preferred embodiment, the TNF-α-induced inflammatory disease that is treated with the composition of the present invention is selected between rheumatoid arthritis, juvenile idiopathic arthritis, psoriatic arthritis, ankylosing spondylitis, Crohn's disease, ulcerative colitis, plaque psoriasis, hidradenitis suppurativa and/or uveitis.

### BRIEF DESCRIPTION OF THE DRAWINGS

FIGS. 1A and 1B show the chromatogram of the psilocin concentration trend in the blank and in the complex of samples A (complex A). FIG. 1A shows the chromatogram of blank psilocin (diluted in H2O:EtOH (8:2). The comparison was made at time 0 and after 24 hours.

FIG. 1B shows the chromatogram of complex 3-carotene:cyclodextrin:ascorbic acid:psilocin. The comparison was made at time 0 and after 24 hours.

FIG. 2 shows the NMR spectra of blank psilocin and complex 3-carotene:cyclodextrin:ascorbic acid:psilocin.

FIG. 3 reports the NMR spectra of samples B to E. FIG. 3A shows the spectra of blank psilocin samples at different stability trials and FIG. 3B shows the spectra of complex samples at different stability trials, both compared to psilocin standard.

FIG. 4 shows the experimental protocol used for the in-vivo test for the treatment of acute inflammation.

FIG. 5 shows the reduction of paw volume following the administration of the complex at 3 and 10 mg/Kg and psilocin at 3 and 10 mg/Kg.

FIG. 6 shows the hematoxylin and eosin (H&E) staining of paw tissues from each experimental group, to estimate histologically the anti-inflammatory effect of psilocin and complex.

FIG. 7 shows that the administrations of psilocin and complex at 3 and 10 mg/Kg significantly increased the paw-withdrawal threshold as compared to CAR group.

### DETAILED DESCRIPTION

The present invention relates to a composition comprising (a) at least one psychedelic non-phosphorylated tryptamine, (b) at least one antioxidant comprising at least one carotenoid, and (c) at least one supplement selected from an amino acid, a vitamin, theobromine, caffeine, resveratrol, gamma aminobutyric acid and combinations thereof.

The at least one psychedelic non-phosphorylated tryptamine of the composition of the invention can be psilocin and/or norpsilocin and/or salts, derivatives, hydrate, or solvate thereof and/or combination thereof.

The at least one psychedelic non-phosphorylated tryptamine can be derived from psychedelic psilocybin mushrooms, which term includes a polyphyletic, informal group of fungi that contain psilocybin, psilocin, or both within their biomass, typically within their fruiting bodies, resulting in their activation of a psychedelic reaction in a subject.

The at least one psychedelic non-phosphorylated tryptamine of the composition of the invention can be preferably derived from psychedelic psilocybin mushrooms belonging to the Psilocybe genus and more preferably it can be psilocin and/or norpsilocin, and/or salts, derivatives, hydrate, or solvate thereof and/or combination thereof. According to a further preferred embodiment, the at least one psychedelic non-phosphorylated tryptamine of the composition of the invention is a combination of psilocin and norpsilocin and/or salts, derivatives, hydrate, or solvate thereof and/or combination thereof.

In a preferred embodiment, the psilocybin-containing mushrooms are of the genus Psilocybe. Non limiting examples of suitable psilocybin-containing mushrooms that are in the genus Psilocybe include Psilocybe atlantis, Psilocybe azurenscens, Psilocybe bohemica, Psilocybe baeocystis, Psilocybe cyanescens, Psilocybe cubensis, Psilocybe tampanensis, Psilocybe hoogshagenii Psilocybe mexicana, Psilocybe ovoideocystidiata, Psilocybe semilanceata, Psilocybe weraroa, Psilocybe stuntzii, Psilocybe cyanofibrillosa, Psilocybe zapotacorum, Psilocybe yungensis, Psilocybe liniformans, Psilocybe xalapensis, Psilocybe venenata, Psilocybe subtropicalis, Psilocybe singer, Psilocybe schultesii, Psilocybe rostrata, Psilocybe quebecensis, Psilocybe pintonii, Psilocybe puberula, Psilocybe mairei, Psilocybe laurae, Psilocybe kumaenorum, Psilocybe beheimii, Psilocybe begalindoi, Psilocybe fmetaria, Psilocybe beegonii, Psilocybe dumontii, Psilocybe carbonaria, Psilocybe cordispora, Psilocybe bispora, Psilocybe aucklandii, and combinations thereof.

The at least one psychoactive non-phosphorylated tryptamine of the composition of the invention can be an extract of psychedelic psilocybin mushrooms, preferably an extract of psychedelic psilocybin mushrooms belonging to the Psilocybe genus, whereas said extract has been purified to contain at least one non-phosphorylated tryptamine, without the presence of phosphorylated tryptamine in the extract.

The at least one antioxidant of the composition of the invention is at least one carotenoid, preferably obtained by plants extraction and/or microbial fermentation.

Particularly preferred carotenoids are a-carotene, β-carotene, lycopene, astaxantin and/or zeaxantine. Even more preferred is β-carotene. The at least one supplement of the composition of the invention is selected from an amino acid, a vitamin, preferably a vitamin of the B6 group or ascorbic acid, a xanthine, such as theobromine and caffeine, a stilbene, such as resveratrol, gamma aminobutyric acid (GABA) and combinations thereof.

Nonlimiting examples of suitable amino acids include tryptophan, arginine, phenylalanine, tyrosine, histidine, arginine, alanine, glycine, serine, threonine, leucine, isoleucine, methionine, valine, theanine, and combinations thereof. In an embodiment, the amino acid is selected from L-tryptophan, L-arginine, L-phenylalanine, L-tyrosine, L-histidine, L-arginine, L-theanine 5-hydroxytryptophan (5-HTTP, also known as oxitriptan), theanine, and combinations thereof.

It is predicted that alternative brain receptor transmitters, such as those replacing or reducing serotonin receptor uptake, and or hallucinogens, such as Ayahuasca, dimethyltryptamine (DMT), and or psilocin/psilocybin extracts or mushroom exposure may be positively enhanced in the presence of selected amino acids (e.g. L-theanine), theobromine, vitamin, in particular vitamin B6, caffeine, resveratrol, gamma aminobutyric acids and combinations thereof; and may more effectively potentiate a positive experience, and further potentiate multiple medical benefits, including but not limited to psychosocial, anti-depressive, panic or post-traumatic stress related, general anxiolytic, and with frequent or daily use facilitate cognitive enhancement, greater amelioration of one or more dementias.

Vitamin B6 is a B vitamin that functions as a coenzyme in enzymatic reactions in metabolism. Nonlimiting examples of vitamin B6 include pyridoxine (PN), pyridoxine 5′-phosphate (P5P), pyridoxal (PL), pyridoxal 5′-phosphate (PLP), pyridoxamine (PM), pyridoxamine 5′-phosphate (PMP), 4-pyridoxic acid (PA), pyritinol, and combinations thereof. In a preferred embodiment, the vitamin B6 is pyridoxine.

In a further preferred embodiment, the vitamin is ascorbic acid. In another preferred embodiment, the supplement is piracetam. Piracetam is also known as 2-oxo-I-pyrrolidine acetamide.

In a preferred embodiment, the supplement is theobromine. Theobromine is also known as 3,7-dimethylxanthine

In a preferred embodiment, the supplement is caffeine. Caffeine is also known as 1,3,7-trimethylxanthine.

In a preferred embodiment, the supplement is resveratrol. Resveratrol is also known as 3,5,4′-trihydroxy stilbene.

In a preferred embodiment, the supplement is gamma aminobutyric acid (GABA). GABA is also known as 4-aminobutanoic acid.

In a further preferred embodiment, the supplement is selected from L-tryptophan, L-arginine, L-phenylalanine, L-tyrosine, L-histidine, L-arginine, 5-HTTP, pyridoxine, piracetam, GABA, theobromine, caffeine, resveratrol, and combinations thereof.

In a further preferred embodiment, the supplement is selected from piracetam, GABA, and combinations thereof.

In a further preferred embodiment, the supplement is selected from theobromine, caffeine, and combinations thereof.

In a further preferred embodiment, the supplement is selected from an amino acid and vitamin B6.

In a further preferred embodiment, the supplement is selected from L-tryptophan, L-arginine, L-phenylalanine, L-tyrosine, L-histidine, L-arginine, 5-HTTP, PN, P5P, PL, PLP, PM, PMP, PA, pyritinol, and combinations thereof.

In a further preferred embodiment, the supplement is selected from L-tryptophan, L-arginine, L-phenylalanine, L-tyrosine, L-histidine, L-arginine, 5-HTTP, PN, and combinations thereof.

In a further preferred embodiment, the supplement is selected from L-tryptophan, L-arginine, L-phenylalanine, 5-HTTP, vitamin B6, piracetam, theobromine, caffeine, GABA, resveratrol, and combinations thereof. In a further preferred embodiment, the supplement is a composition containing, consisting essentially of, or consisting of L-tryptophan, L-arginine, L-phenylalanine, 5-HTTP, vitamin B6, piracetam, theobromine, caffeine, GABA, and resveratrol.

Optionally, the composition of the invention further comprises at least one solubilization agent.

Hence, according to a preferred embodiment, the composition of the invention comprises at least one psychedelic non-phosphorylated tryptamine, at least one antioxidant, wherein the antioxidant is at least one carotenoid, at least one supplement selected from an amino acid, a vitamin, a xanthine, a stilbene, gamma aminobutyric acid and combinations thereof, and at least one solubilization agent.

According to another preferred embodiment, the composition of the invention comprises at least one psychedelic non-phosphorylated tryptamine, at least one antioxidant, wherein the antioxidant is at least one carotenoid, at least one supplement selected from an amino acid, a vitamin, a xanthine, a stilbene, gamma aminobutyric acid and combinations thereof, and at least one solubilization agent, wherein the at least one psychedelic non-phosphorylated tryptamine, the at least one antioxidant, wherein the antioxidant is at least one carotenoid, the at least one supplement selected from an amino acid, a vitamin, a xanthine, a stilbene, gamma aminobutyric acid and combinations thereof, and the at least one solubilization agent are in the form of an inclusion complex.

Preferably, the ratio of the at least one psychedelic non-phosphorylated tryptamine, the at least one antioxidant, wherein the antioxidant is at least one carotenoid and the at least one supplement selected from an amino acid, a vitamin, a xanthine, a stilbene, gamma aminobutyric acid and combinations thereof, is 1:1:1 in the inclusion complex comprised in the composition of the invention.

Preferably, the at least one solubilization agent is at least one cyclodextrin, and more preferably said at least one cyclodextrin is selected from a-cyclodextrin or derivatives thereof, β-cyclodextrin or derivatives thereof or y-cyclodextrin or derivatives thereof.

According to a further preferred embodiment, β-cyclodextrins or derivatives thereof are particularly preferred.

A β-cyclodextrin derivative which is particularly preferred is a hydroxyalkyl-β-cyclodextrin, such as 2-hydroxypropylether β-cyclodextrin (hydroxypropyl-β-cyclodextrin).

According to a further preferred embodiment, the composition of the invention comprises at least one psychedelic non-phosphorylated tryptamine, at least one antioxidant, wherein the antioxidant is at least one carotenoid, at least one supplement selected from a vitamin, and at least one solubilization agent, optionally in the form of an inclusion complex.

According to a particularly preferred embodiment, the composition of the invention comprises psilocin, β-carotene, ascorbic acid, and at least one solubilization agent, optionally in the form of an inclusion complex.

According to a particularly preferred embodiment, the stabilized composition of the invention comprises psilocin, β-carotene, ascorbic acid, and at least one cyclodextrin, preferably a a-cyclodextrin or derivatives thereof, 3-cyclodextrin or derivatives thereof or y-cyclodextrin or derivatives thereof, optionally in the form of an inclusion complex.

According to a particularly preferred embodiment, the composition of the invention comprises psilocin, β-carotene, ascorbic acid, and 2-hydroxypropylether β-cyclodextrin.

According to another particularly preferred embodiment, the composition of the invention comprises psilocin, β-carotene, ascorbic acid, and 2-hydroxypropylether β-cyclodextrin in the form of an inclusion complex. Optionally, the composition of the invention can further comprise at least one MAOI compound.

According to a preferred embodiment, the at least one MAOI compound of the composition of the invention belongs to the 3-carboline class of inhibitors. Preferably, the at least one MAOIs compound belonging to the 3-carboline class of inhibitors is selected from harman, harmine, norharmane, harmol, 6-methoxyharmalan, harmalan, harmaline, harmalol, dihydro-p-carbolines (DHpC), tetrahydro-p-carboline (THpC), tetrahydroharmine, methyl-tetrahydro-p-carboline MTHpC, pinoline, 1-trichloromethyl-1,2,3,4tetrahydro-b-carboline (TaClo), 6-methoxytetrahydroharmalan, ethyl P-carboline-3-carboxylate (PCCE), p-carboline-3-carboxylate (PCCM), manzamine A, manzamine X, 6-deoxymanzamine X, manzamine Y, 8-hydroxymanzamine A, 8-methoxymanzamine A, 6-hydroxymanzamine A, 3,4-dihydromanzamine A, ent-8-hydroxymanzamine A, ent-manzamine F, neo-kauluamine, xestomanzamine B, hyrtioerectines A, gesashidine A, plakortamines A, plakortamines B, plakortamines D, plakortamines C, eudistomidins, threctandramine, fascaplysin and/or combination thereof.

According to a preferred embodiment, the at least one MAOI compound belonging to the β-carboline class of inhibitors is selected from harman, harmine, norharmane, harmol, 6-methoxyharmalan, harmalan, harmaline and/or harmalol.

The preferred molar ratio between the at least one psychedelic non-phosphorylated tryptamine and the at least one antioxidant, wherein the antioxidant is at least one carotenoid, of the composition of the invention is comprised between about 10:1 to about 1:10.

In one embodiment, the composition disclosed herein comprise a molar ratio between about 100:1 to about 1:100 of the at least one psychedelic non-phosphorylated tryptamine and of the at least one antioxidant, wherein the antioxidant is at least one carotenoid.

In another embodiment, the compositions disclosed herein comprise a molar ratio between about 1,000:1 to about 1:1,000 of the at least one psychedelic non-phosphorylated tryptamine and of the at least one antioxidant.

In one embodiment, the compositions disclosed herein comprise a molar ratio of about 10,000:1 to about 1:10,000 of the at least one psychedelic non-phosphorylated tryptamine and of the at least one antioxidant, wherein the antioxidant is at least one carotenoid.

A further object of the invention relates the composition of at least one psychedelic non-phosphorylated tryptamine, at least one antioxidant, wherein the antioxidant is at least one carotenoid, and at least one supplement selected from an amino acid, a vitamin, theobromine, caffeine, resveratrol, gamma aminobutyric acid and combinations thereof, for use in the treatment of fibromyalgia, spinal cord injury-induced chronic neuropathic pain, neuropathic pain associated with diabetic peripheral neuropathy, post-herpetic neuralgia, chronic musculoskeletal pain and/or a TNF-α-induced inflammatory disease. Therapeutically useful amounts of the composition of the present invention generally fall within a daily administration dose range of 0.01-1000 mg.

According to a preferred embodiment, the TNF-α-induced inflammatory disease is selected between rheumatoid arthritis, juvenile idiopathic arthritis, psoriatic arthritis, ankylosing spondylitis, Crohn's disease, ulcerative colitis, plaque psoriasis, hidradenitis suppurativa and/or uveitis.

A further object of the invention relates to an inclusion complex of at least one psychedelic non-phosphorylated tryptamine, at least one antioxidant, wherein the antioxidant is at least one carotenoid, at least one supplement selected from an amino acid, a vitamin, a xanthine, a stilbene, gamma aminobutyric acid and combinations thereof, and at least one solubilization agent.

Preferably, the ratio of the at least one psychedelic non-phosphorylated tryptamine, the at least one antioxidant, wherein the antioxidant is at least one carotenoid, and the at least one supplement selected from an amino acid, a vitamin, a xanthine, a stilbene, gamma aminobutyric acid and combinations thereof, is 1:1:1 in the inclusion complex of the invention.

According to a preferred embodiment in the inclusion complex of the invention:
the at least one psychedelic non-phosphorylated tryptamine is psilocin and/or norpsilocin and/or salts, derivatives, hydrate, or solvate thereof and/or combination thereof; and/or—the at least one antioxidant, wherein the antioxidant is at least one carotenoid selected from α-carotene, β-carotene, lycopene, astaxantin and/or zeaxantine; and/or
the at least one supplement selected from an amino acid, a vitamin, a xanthine, a stilbene, gamma aminobutyric acid and combinations thereof, is an amino acid preferably selected from tryptophan, arginine, phenylalanine, tyrosine, histidine, arginine, alanine, glycine, serine, threonine, leucine, isoleucine, methionine, valine, theanine, and combinations thereof; and/or is a vitamin, preferably a vitamin B6 or ascorbic acid, and more preferably the vitamin B6 is selected from pyridoxine, pyridoxine 5′-phosphate, pyridoxal, pyridoxal 5′-phosphate, pyridoxamine (PM), pyridoxamine 5′-phosphate (PMP), 4-pyridoxic acid (PA), pyritinol, and combinations thereof; and/or is a xanthine, preferably theobromine, caffeine and/or combination thereof; and/or is a stilbene, preferably resveratrol; and/or is gamma aminobutyric acid;
the at least one solubilization agent is at least one cyclodextrin, and more preferably said at least one cyclodextrin is selected from a-cyclodextrin or derivatives thereof, β-cyclodextrin or derivatives thereof or γ-cyclodextrin or derivatives thereof. According to a further preferred embodiment, β-cyclodextrins or derivatives thereof are particularly preferred. A β-cyclodextrin derivative which is particularly preferred is a hydroxyalkyl-β-cyclodextrin, such as 2-hydroxypropylether β-cyclodextrin (hydroxypropyl-β-cyclodextrin).

According to a particularly preferred embodiment, the inclusion complex of the invention is an inclusion complex of at least one psychedelic non-phosphorylated tryptamine, at least one antioxidant, wherein the antioxidant is at least one carotenoid, at least one supplement selected from a vitamin, and at least one solubilization agent.

According to a more preferred embodiment, the inclusion complex of the invention is an inclusion complex of psilocin, β-carotene, ascorbic acid, and at least one solubilization agent. According to a furthermore preferred embodiment, the inclusion complex of the invention is an inclusion complex of psilocin, β-carotene, ascorbic acid, and at least one cyclodextrin, preferably a a-cyclodextrin or derivatives thereof, β-cyclodextrin or derivatives thereof or y-cyclodextrin or derivatives thereof.

According to a furthermore preferred embodiment, the inclusion complex of the invention is an inclusion complex of psilocin, β-carotene, ascorbic acid, and 2-hydroxypropylether β-cyclodextrin.

Preferably, the ratio of psilocin, β-carotene, and ascorbic acid is 1:1:1.

The inclusion complex according to the invention can be obtained using a process comprising the following steps:
1) dissolving the solubilization agent in water;
2) dissolving the at least one antioxidant, wherein the antioxidant is at least one carotenoid, in a polar solvent, preferably an alcohol, more preferably ethyl alcohol, thereby obtaining an antioxidant solution;
3) adding the antioxidant solution to the solubilization agent solution under continuous stirring until a clear solution is obtained;
4) adding the at least one supplement selected from an amino acid, a vitamin, a xanthine, a stilbene, gamma aminobutyric acid and combinations thereof to the solution obtained at point 3) and freeze, preferably at −80° C., and then freeze-dry the solution to obtain a complex;
5) dissolving the at least one psychedelic non-phosphorylated tryptamine in an aqueous alcohol solution, preferably an aqueous ethyl alcohol solution, and use the obtained suspension to resuspend the freeze-dried complex, thus obtaining the inclusion complex.

Preferably, the aqueous alcohol solution is a 20% alcohol solution, more preferably is a 20% ethyl alcohol solution.

Alternatively, step 5) can be performed as follows:
5a) dissolving the at least one psychedelic non-phosphorylated tryptamine in an aqueous alcohol solution, preferably an aqueous ethyl alcohol solution. Dissolving the solubilization agent in water at room temperature and mixing with the aqueous alcohol solution containing the at least one psychedelic non-phosphorylated tryptamine. Freeze, preferably at −80° C., and then freeze-dry the solution. Then, re-suspend the obtained freeze-dried solution in water and add to the freeze-dried complex obtained from step 4), thus obtaining the inclusion complex.

According to a preferred aspect, the ratio between the antioxidant, wherein the antioxidant is at least one carotenoid, and the solubilization agent is between 1:1 and 1:5, preferably 1:1, 1:3, or 1:5, and more preferably a ratio of 1:5.

### EXAMPLES

### Example 1—Preparation of an Inclusion Complex of Psilocin, β-Carotene, Ascorbic Acid, and 2-hydroxypropylether β-Cyclodextrin

Firstly, a complex of β-carotene and ascorbic acid (1:1) is prepared, using 2-hydroxypropylether β-cyclodextrin as solubilization agent. Different ratios of β-carotene to 2-hydroxypropylether β-cyclodextrin were used: 1:1; 1:3; and 1:5. The ratio of 1:5 is particularly preferred.

Ratio 1:1 39 mg of 2-hydroxypropylether β-cyclodextrin were dissolved in 7 mL of water; 3 mg (1.5 pl) of β-carotene were dissolved in 1.4 mL of EtOH. β-carotene solution was added slowly to the cyclodextrin solution under continuous stirring until a clear solution was obtained. Finally, 3 mg of ascorbic acid were added to the solution, and it was mixed for 10 minutes, frozen at −80° C. and finally freeze-dried.

Ratio 1:3:1 16 mg of 2-hydroxypropylether β-cyclodextrin were dissolved in 20 mL of water; 3 mg (1.5 pl) of β-carotene were dissolved in 4 mL of EtOH. β-carotene solution was added slowly to the cyclodextrin solution under continuous stirring until a clear solution was obtained. Finally, 3 mg of ascorbic acid were added to the solution, and it was mixed for 10 minutes, frozen at −80° C., and finally freeze-dried.

Ratio 1:5 193 mg of 2-hydroxypropylether β-cyclodextrin were dissolved in 33 mL of water; 3 mg (1.5 pl) of β-carotene were dissolved in 7 mL of EtOH. β-carotene solution was added slowly to the cyclodextrin solution under continuous stirring until a clear solution was obtained. Finally, 3 mg of ascorbic acid were added to the solution, and it was mixed for 10 minutes, frozen at −80° C. and finally freeze-dried.

3 mg of psilocin was dissolved in 1 mL of H₂O:EtOH (8:2) solution and it was used for resuspending the freeze-dried complexes of p-carotene and ascorbic acid obtained, thus obtaining the inclusion complex comprising also psilocin.

### Example 2

### Stability Tests

The inclusion complex obtained by the resuspension of the freeze-dried complex of (β-carotene and 2-hydroxypropylether p-cyclodextrin in the ratio 1:5 with 3 mg of psilocin dissolved in 1 mL of H₂O:EtOH (8:2) solution, was tested to evaluate the inclusion complex stability.

The complex stability was evaluated for 24 hours, under different environmental conditions:
Sample A) 22° C. with no light irradiation
Sample B) 30° C. with no light irradiation
Sample C) 22° C. under UV irradiation (using an UVA lamp A=365 nm)
Sample D) Freeze-dried for 30 days
Sample E) 30° C. under UV irradiation (using an UVA lamp A=365 nm)

All the complex stability tests were compared to a “blank” psilocin solution in H₂O:EtOH (8:2).

For evaluating psilocin stability chromatographic analyses were performed using a Nexera X2 UHPLC system coupled to a LCMS-8060 mass spectrometer (Shimadzu, Duisburg, Germany). Data acquisition and processing was performed using the LabSolutions software (version 5.95 Shimadzu). The separation of the various tryptamines was achieved by using an Ascentis Express 90 HILIC column (150×2.1 mm, 2.7 pm) (Merck Life Science) and a mobile phase composed of ammonium formate 0.1 M in water as solvent A, and acetonitrile as solvent B. The gradient program was as follows: 0 min, 90% B; 3 min, 75% B; 10 min, 20% B; 13 min 20% B; 15 min 90% B. The flow rate was 0.5 mL min−1; the column oven was set at 40° C. and the injection volume was 1 pL. Mobile phases were filtered through a 0.45 pm membrane.

### Nuclear Magnetic Resonance

The same specimens were analyzed also by NMR Spectroscopy using Varian 500 MHz spectrometer equipped with a OneNMR probe.

Samples were recorded using a PRESAT pulse sequence suppressing the signal of H₂O and EtOH acquiring 128 scans for each spectrum. H₂O (4.64 ppm) was used as reference line.

For the NMR analysis, all samples were treated as follow: 530 pL were poured into NMR tube (5 mm i.d.) then coaxial tube was filled with D2O to lock the solvent frequency.

SAMPLES A: Stability test carried out at 22° C. with no light irradiation No darkening was observed, after 24 h, for the complex of 3-carotene:cyclodextrin:ascorbic acid:psilocin stored at 22° C. with no light irradiation. On the contrary, the blank solution, represented by psilocin with no complex, started the darkening process since the first 2 hours of storing.

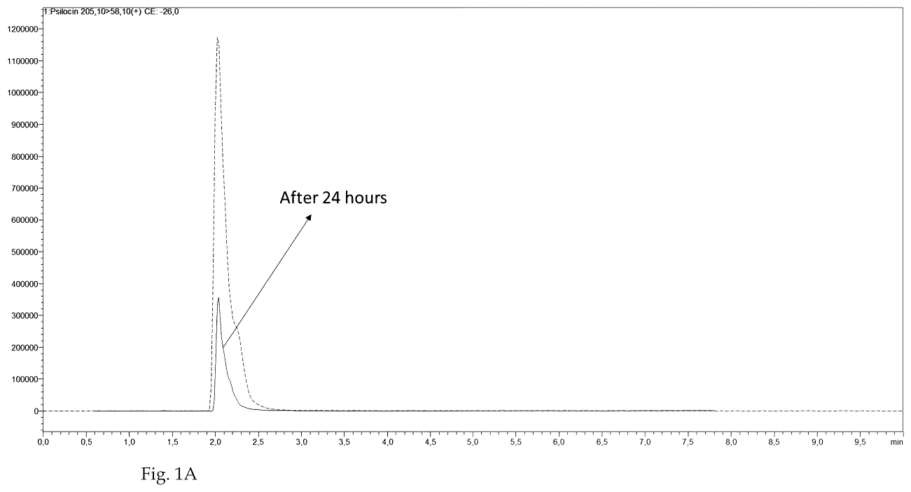

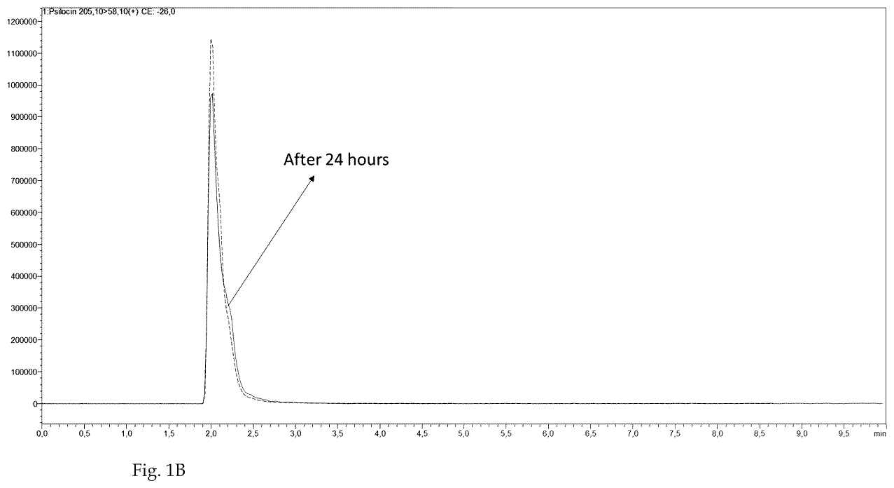

Psilocin was evaluated both for blank and for the complex at time 0 and after 24 hours. In the blank sample the psilocin concentration decreased. On the contrary, in the complex sample the concentration was stable during the 24 hours. FIGS. 1A and 1B show the chromatogram regarding the psilocin concentration trend in the blank and in the complex of samples A (complex A).

The synthetic psilocin was weighed in a volumetric flask and diluted with water: ethanol (8:2) at a concentration of 3000 ppm. Then, it was serially diluted to get a final concentration of 0.10 ppm and analyzed according to the above reported method, in order to verify the stability for 24 hours. At time 0, it was 0.11 ppm. After 24 hours the concentration decreased to 0.04 ppm, showing a concentration loss of 60%.

On the contrary, just a 20% loss of psilocin concentration was detected when the complex of the present invention was employed. It showed an initial psilocin concentration at time 0 of 0.10 ppm, and this concentration decreased to 0.08 ppm after 24 hours.

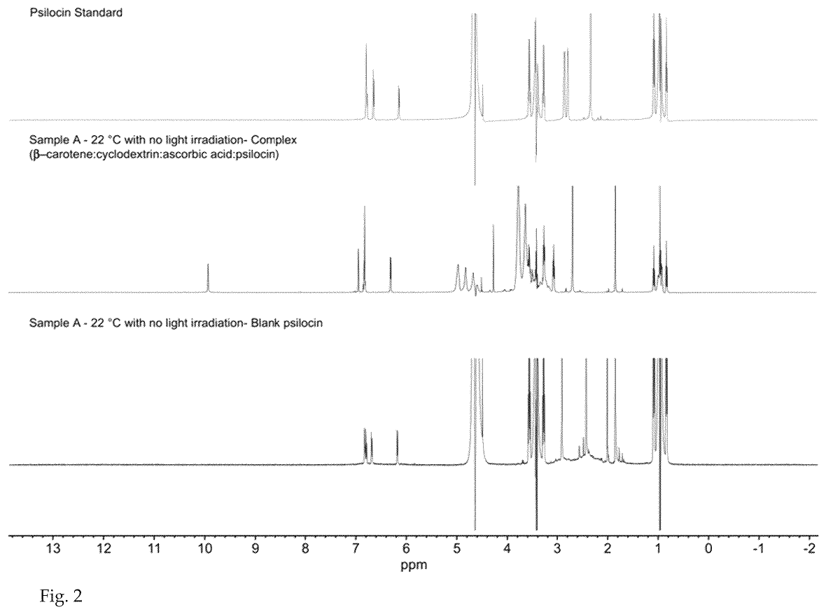

A single set of aromatic signals were detected in the complex. Moreover, it is possible to detect a signal of around 10 ppm ascribable to indole NH. Both samples, complex and blank, present less degradation product respect to the other solutions treated in different conditions. In FIG. 2 are shown NMR spectra of blank and complex in comparison with psilocin standard.

SAMPLES B: Stability test carried out at 30° C. with no light irradiation. The darkening process was observed just for the blank solution. On the other hand, the psilocin concentration decreased during 24 hours for both the samples, blank and complex. This behavior can be ascribable to the temperature effect.

SAMPLES C: No darkening was observed after 24 h for the complex stored at 22° C. under UV irradiation. On the contrary, the blank solution, represented by psilocin with no complex, started the darkening process since the first 2 hours of storing.

Psilocin was evaluated both for blank and for the complex at time 0 and after 24 hours. The psilocin concentration was decreasing during 24 hours for both the samples. This behavior can be ascribable to the UV irradiation effect.

SAMPLES D: The stability test was carried out at ambient temperature on freeze-dried samples. The samples were redissolved 30 days after freeze-drying. No darkening was observed, after 24 h, for the complex stored at ambient temperature. On the contrary, the blank solution, represented by psilocin with no complex, started the darkening process since the first 2 hours of storing. The results were not repeatable, this behavior can be due because the psilocin can be damaged by freeze-thaw cycles.

SAMPLES E: Stability test carried out at 30° C. under UV irradiation.

Darkening was observed, after 24 h, for both samples, blank and complex solutions. Moreover, the psilocin concentration decreased during 24 hours for both the samples. This behavior can be due because the psilocin can be damaged by the combination of temperature and light irradiation.

The combinate results obtained from LC-MS/MS and NMR Spectroscopy suggest that the samples (B, C, D and E) subjected to different light and temperature conditions were characterized by a decreasing psilocin concentration for either complex or blank. Nevertheless, the psilocin decreasing was lower when the complex was employed.

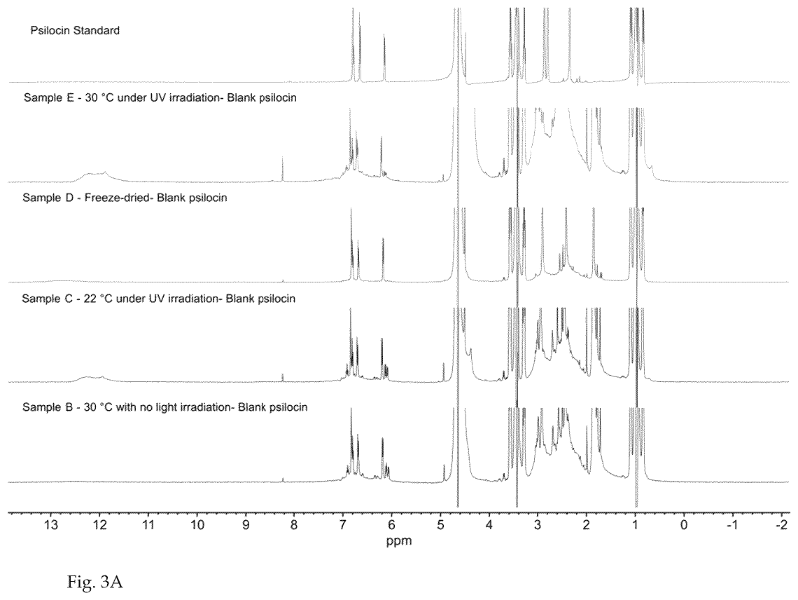

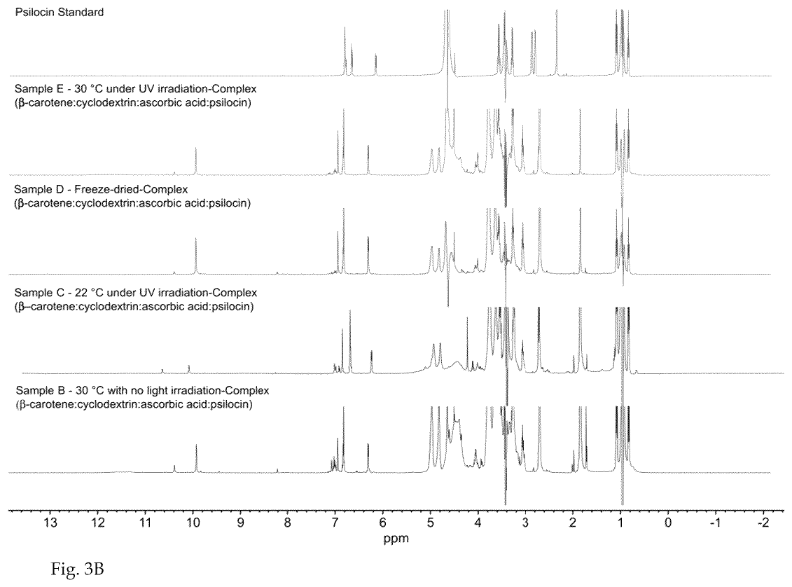

FIG. 3 reports the NMR spectra of samples B to E. In FIG. 3A are shown spectra of blanks and in FIG. 3B the spectra of complexes, both compared to psilocin standard.

Finally, according to NMR analysis the tested complexes show a protective effect against the degradation of the psilocin, since, less aromatic signals, not ascribable to indole moiety, are detected.

According to the obtained results for stability, the complex A was able to protect the psilocin against the oxidation, due to air and light for 24 hours at 22° C.

It is possible to point out that the best storage conditions are a temperature of not higher than 22° C. with no UV irradiation.

### Example 3

### Experimental In-Vivo Test for the Treatment of Acute Inflammation

The biological effect of the complex, in comparison with the psilocin solution, against inflammation was tested by their administration to 8-12 weeks old male rats.

A composition comprising complex according to example 1, wherein the ratio of β-carotene to 2-hydroxypropylether β-cyclodextrin was 1:5, was used. Complexes (psilocin, β-carotene, ascorbic acid with ratio 1:1:1 and 2-hydroxypropylether β-cyclodextrin) containing two different concentrations of psilocin have been tested:
a complex containing 3 mg of psilocin,
a complex containing 10 mg of psilocin.

### Experimental Protocol

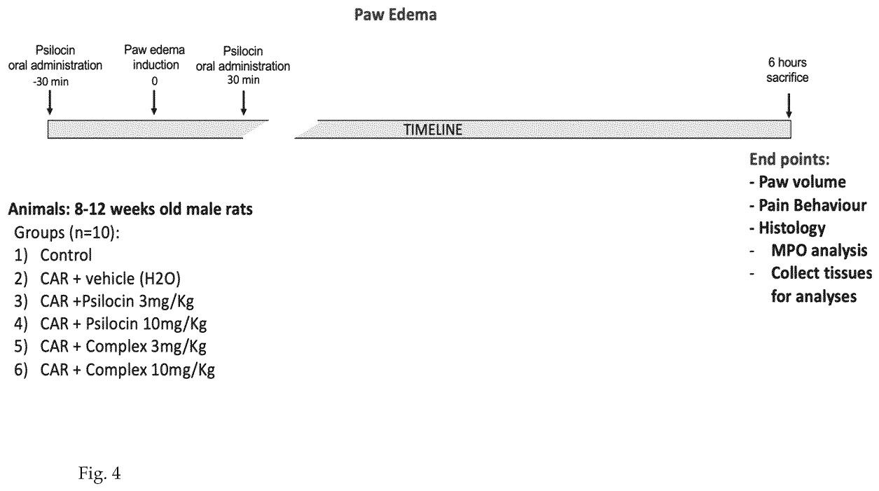

FIG. 4 shows the experimental protocol.

The study was performed using male Sprague-Dawley rats (200-250 g; Envigo, Milan, Italy). Food and water were accessible ad libitum. Rats were concurrently subjected to a singular subplantar injection of CAR (100 pL of saline solution containing 1% CAR). Paw edema was measured with a plethysmometer (Ugo Basile, Comerio, Varese, Italy) (Salvemini, D.; Wang, Z.-Q.; Wyatt, P. S.; Bourdon, D. M.; Marino, M. H.; Manning, P. T.; Currie, M. G., “Nitric oxide: A key mediator in the early and late phase of carrageenan-induced rat paw inflammation”, Br. J. Pharmacol. 1996, 118, 829-838) prior to CAR injection and every hour for 6 h. Edema was expressed as the increase in paw volume (mL) after CAR injection relative to the pre-injection value for all animals. Scores were expressed as paw volume difference (mL).

### Pain Behavior

Mechanical allodynia was evaluated using a dynamic plantar Von Frey hair aesthesiometer on time 0 and 1, 2, 3, 4, 5, and 6 hours post-injection (Bio-EVF4; Bioseb, Vitrolles, France) as previously described (D'Amico, R.; Fusco, R.; Siracusa, R.; Impellizzeri, D.; Peritore, A. F.; Gugliandolo, E.; Interdonato, L.; Sforza, A. M.; Crupi, R.; Cuzzocrea, S.; et al., “Inhibition of P2X7 Purinergic Receptor Ameliorates Fibromyalgia Syndrome by Suppressing NLRP3 Pathway.”, Int. J. Mol. Sci. 2021, 22, 6471. https://doi.org/10.3390/ijms22126471). The device encloses a force transducer furnished with a plastic tip. When pressure is applied to the tip, the force applied is recorded. The tip was applied to the plantar area of the hind leg, and a rising upward force was exerted until the paw was withdrawn. The withdrawal threshold was defined as the force, expressed in grams, at which the mouse removed the paw.

### Histology

Paw tissues were collected 6 h after CAR injection. Samples were fixed in 10% formaldehyde solution in phosphate buffer saline (PBS) at room temperature for 24 h, dehydrated by graded series of ethanol and at the list embedded in Paraplast (Sherwood Medical, Mahwah, NJ, USA). Sections of 7-pm thickness were cut with a microtome, deparaffinized with xylene and stained with hematoxylin and eosin (H&E). All sections were examined using an Axiovision Zeiss (Milan, Italy) microscope by two investigators blinded to the treatment conditions. The histological analysis was performed according to a previously described method (Petrosino, S.; Cordaro, M.; Verde, R.; Moriello, A. S.; Marcolongo, G.; Schievano, C.; Siracusa, R.; Piscitelli, F.; Peritore, A. F.; Crupi, R.; et al., “Oral Ultramicronized Palmitoylethanolamide: Plasma and Tissue Levels and Spinal Anti-hyperalgesic Effect”, Front. Pharmacol. 2018, 9, 2) and given the following score from 0 to 5:0=no inflammation, 1=mild inflammation, 2=mild/moderate inflammation, 3=moderate inflammation, 4=moderate/severe inflammation and 5=severe inflammation.

### MPO Analysis

The activity of MPO (myeloperoxidase is an enzyme released by neutrophils and used as a marker of neutrophil infiltration) was assessed as previously described (Mullane, K. M.; Kraemer, R.; Smith, B. “Myeloperoxidase activity as a quantitative assessment of neutrophil infiltration into ischemic myocardium.” J. Pharmacol. Methods 1985, 14, 157-167). The rate of change in absorbance was calculated spectrophotometrically at 650 nm. MPO activity was measured in U per gram weight of wet tissue and was quantified as the quantity of enzyme degrading 1 pmol of peroxide 1 min at 37° C.

### Statistical Analysis

All values are expressed as mean±standard deviation (SD) of N=10 observations. For the in-vivo studies, N represents the number of animals studied. Data were analyzed by one-way ANOVA followed by a Bonferroni post-hoc test for multiple comparisons. A p-value less than 0.05 will be considered significant. #p<0.05 vs. CTR, *p<0.05 vs. CAR, ##p<0.01 vs. CTR, **p<0.01 vs. CAR, ###p<0.001 vs. CTR, ***p<0.001 vs. CAR, °p<0.05 vs. 3 m/Kg, § p<0.05 vs. 10 mg/Kg, °°p<0.01 vs. 3 mg/Kg, §§ p<0.01 vs. 10 mg/Kg, 000 p<0.001 vs. 3 mg/Kg, §§§ p<0.001 vs. 10 mg/Kg.

### Edema Volume

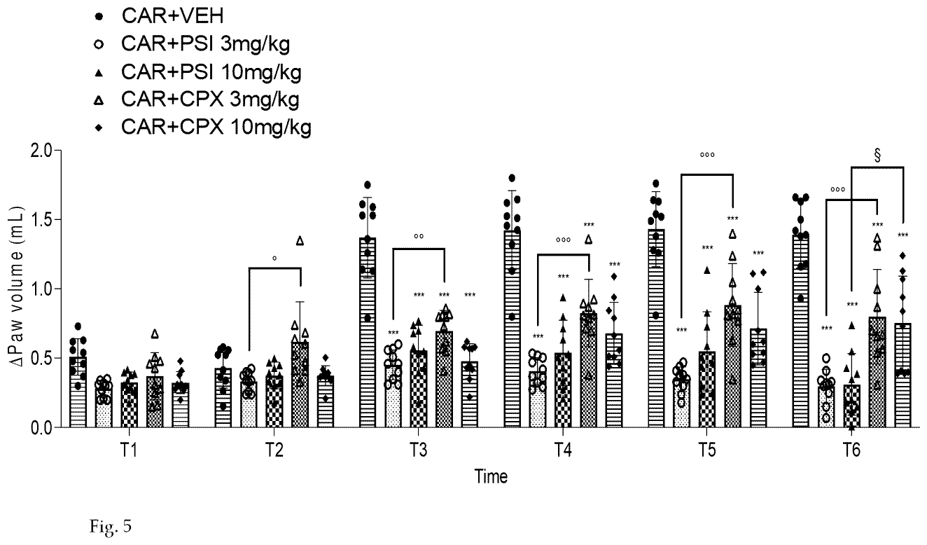

Injection of CAR into the hind paw of rats led to a significant time-dependent increase in paw volume. CAR-induced paw edema was significantly reduced by treatment with Psilocin at 3 and 10 mg/Kg. The administration of complex at 3 and 10 mg/Kg also reduced the paw volume, as can be seen in FIG. 5. Moreover, psilocin administration at all doses showed a significant effect as compared to the complex administration.

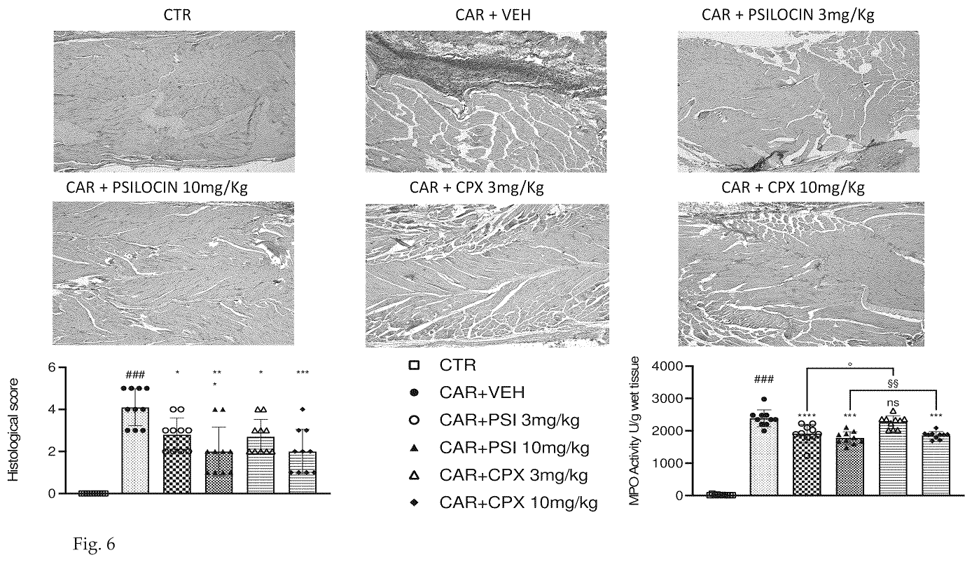

In order to estimate histologically the anti-inflammatory effect of psilocin and complex, paw tissues from each experimental group were observed by hematoxylin and eosin (H&E) staining, as reported in FIG. 6. No histologic damage was detected in control rats. In contrast, CAR injection into the hind paw caused a notable accumulation of infiltrating inflammatory cells as compared to control group. Inflammatory cell infiltration and edema were meaningfully decreased with psilocin administration at 3 and 10 mg/Kg, but the highest dose worked better.

Additionally, complex administration followed the same trend, in particular, its administration at 10 mg/Kg strongly decreased the histological score, as compared to the 3 mg/Kg administration. Moreover, psilocin administration at all doses showed a better anti-inflammatory effect as compared to the complex administration. The development of histological injury was accompanied by increased infiltration of neutrophils, as revealed by an increase in MPO activity. In the CAR group the MPO activity was significantly increased, as compared to control. Psilocin administration at 3 and 10 mg/Kg reduced the MPO activity, and the complex administration at 10 mg/Kg obtained the same effect as well. Moreover, psilocin administration at all doses showed a significant effect, as compared to the complex administration.

### Pain Behavior

To investigate whether psilocin and complex were able to reduce pain associated with CAR injection, Von Frey test was performed. No significant differences were observed in mechanical hyperalgesia in all animals at time 0. CAR injection reduced the paw-withdrawal threshold in response to von Frey hair stimulation at the other timepoints.

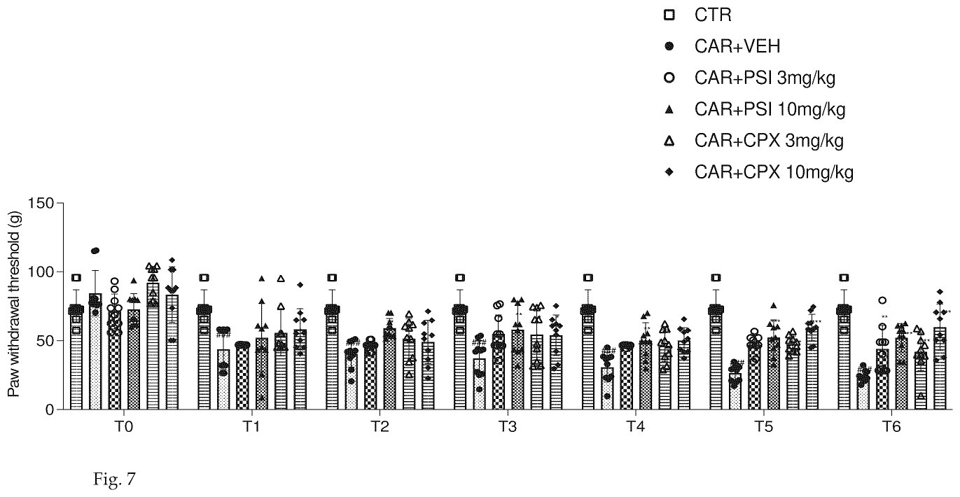

Administrations of psilocin and complex at 3 and 10 mg/Kg significantly increased the paw-withdrawal threshold as compared to CAR group, as shown in FIG. 7. Thus, both compounds showed analgesic effects.

1. A composition comprising (a) at least one psychedelic non-phosphorylated tryptamine, (b) at least one antioxidant comprising is at least one carotenoid, and (c) at least one supplement selected from an amino acid, a vitamin, a xanthine, a stilbene, gamma aminobutyric acid and combinations thereof.

2-19. (canceled)

20. The composition of claim 1, wherein the at least one psychedelic non-phosphorylated tryptamine is psilocin, norpsilocin, salts, derivatives, hydrates, or solvates thereof, and combination thereof.

21. The composition according to claim 1, wherein the at least one psychedelic non-phosphorylated tryptamine is a combination of psilocin and norpsilocin, salts, derivatives, hydrates, or solvates thereof, and combinations thereof.

22. The composition according to claim 1, wherein the at least one psychedelic non-phosphorylated tryptamine is derived from psychedelic psilocybin mushrooms belonging to the Psilocybe genus.

23. The composition according to claim 1, wherein the at least one psychedelic non-phosphorylated tryptamine is an extract of psychedelic psilocybin mushrooms, preferably an extract of psychedelic psilocybin mushrooms belonging to the Psilocybe genus, whereas said extract has been purified to contain at least one non-phosphorylated tryptamine, without the presence of phosphorylated tryptamine.

24. The composition according to claim 1, wherein the at least one antioxidant is at least one carotenoid extract or fermentation product.

25. The composition according to claim 1, wherein the at least one antioxidant is at least one carotenoid selected from a-carotene, p-carotene, lycopene, astaxantin and/or zeaxantine.

26. The composition according to claim 1, wherein the at least one supplement is an amino acid, preferably selected from tryptophan, arginine, phenylalanine, tyrosine, histidine, arginine, alanine, glycine, serine, threonine, leucine, isoleucine, methionine, valine, theanine, and combinations thereof.

27. The composition according to claim 1, wherein the at least one supplement is a vitamin

28. The composition according to claim 1, wherein the at least one supplement is a xanthine.

29. The composition according to claim 1, wherein the at least one supplement is a stilbene.

30. The composition according to claim 1, further comprising at least one solubilization agent.

31. The composition according to claim 12, wherein the antioxidant comprises at least one carotenoid; the at least one supplement is an amino acid, a vitamin, a xanthine, a stilbene, gamma aminobutyric acid, and combinations thereof; and the composition is in the form of an inclusion complex.

32. The composition according to claim 1, further comprising at least one MAOI compound, wherein the at least one MAOI compound is preferably selected from harman, harmine, norharmane, harmol, 6-methoxyharmalan, harmalan, harmaline, harmalol, dihydro-|3-carbolines (DH3C), tetrahydro-3-carboline (TH3C), tetrahydroharmine, methyl-tetrahydro-3-carboline MTH3C, pinoline, 1-trichloromethyl-1,2,3,4tetrahydro-b-carboline (TaClo), 6-methoxytetrahydroharmalan, ethyl P-carboline-3-carboxylate (3CCE), 3-carboline-3-carboxylate (3CCM), manzamine A, manzamine X, 6-deoxymanzamine X, manzamine Y, 8-hydroxymanzamine A, 8-methoxymanzamine A, 6-hydroxymanzamine A, 3,4-dihydromanzamine A, ent-8-hydroxymanzamine A, ent-manzamine F, neo-kauluamine, xestomanzamine B, hyrtioerectines A, gesashidine A, plakortamines A, plakortamines B, plakortamines D, plakortamines C, eudistomidins, threctandramine, fascaplysin and/or combination thereof.

33. The composition according to claim 14 wherein the at least one MAOI compound is selected from harman, harmine, norharmane, harmol, 6-methoxyharmalan, harmalan, harmaline, harmalol and/or combination thereof.

34. The composition according to claim 1, wherein the molar ratio between the at least one non-phosphorylated tryptamine and the at least one antioxidant is between about 10:1 to about 1:10.

35. A stabilized psilocin composition according to claim 1, that is therapeutically useful to treat a patient suffering from fibromyalgia, spinal cord injury-induced chronic neuropathic pain, neuropathic pain associated with diabetic peripheral neuropathy, post-herpetic neuralgia, chronic musculoskeletal pain and/or a TNF-α-induced inflammatory disease.

36. The composition according to claim 17, wherein the TNF-α-induced inflammatory disease is selected between rheumatoid arthritis, juvenile idiopathic arthritis, psoriatic arthritis, ankylosing spondylitis, Crohn's disease, ulcerative colitis, plaque psoriasis, hidradenitis suppurativa and/or uveitis.

37. The composition according to claim 9 wherein said vitamin comprises vitamin B6 or ascorbic acid.

38. An inclusion complex comprising: (a) at least one psychedelic that is not a phosphorylated tryptamine, (b) at least one antioxidant wherein the antioxidant comprises at least one carotenoid, (c) at least one supplement selected from an amino acid, a vitamin, a xanthine, a stilbene, gamma aminobutyric acid and combinations thereof, and (d) at least one solubilization agent.
